(19) United States
(12) Patent Application Publication
(10) Pub. No.: US 2012/0215135 A1
(43) Pub. Date: Aug. 23, 2012

(54) Pelvic Region Analyzer and Associated Therapeutic and Diagnostic Methods

(76) Inventors: Domingo E. Galliano, Port Charlotte, FL (US); Kathleen Lueck, Port Charlotte, FL (US); Christopher D. Ross, Davie, FL (US); Tomas Fernandez, Miramar, FL (US)

(21) Appl. No.: 13/399,906

(22) Filed: Feb. 17, 2012

Related U.S. Application Data
(60) Provisional application No. 61/443,759, filed on Feb. 17, 2011.

(51) Int. Cl. A61B 5/11; A61B 5/103

(52) U.S. Cl. 600/595; 600/587

(57) ABSTRACT

A pelvic region analyzer for performs at least one of therapeutic and diagnostic methods. The pelvic region analyzer has a probe for insertion in a pelvic region of a patient. The pelvic region analyzer has a balloon for insertion in the pelvic region and disposed at a distal end of the probe. A sensor is integrated with the probe and disposed at a distal end of the probe. An electronic coupling is coupled to the sensor. The electronic coupling provides pressure information from the sensor. An air tube is connected to the balloon. Air is added and removed from the air tube in accordance with at least one of the therapeutic and diagnostic methods.

### CROSS-REFERENCE TO RELATED APPLICATIONS

This application claims priority to U.S. Provisional Application Ser. No. 61/443,759, filed on Feb. 17, 2011, the entire disclosure of which is hereby incorporated herein by reference in its entirety.

### STATEMENT REGARDING FEDERALLY SPONSORED RESEARCH OR DEVELOPMENT

Not Applicable

### FIELD OF THE INVENTION

The present disclosure lies in the field of pelvic floor dysfunction. The present disclosure relates to methods and apparatuses/devices for providing pelvic floor dysfunction therapeutics and diagnostics.

### BACKGROUND OF THE INVENTION

Pelvic floor dysfunction, e.g. urinary or anal incontinence, constipation, and pelvic pain, may be treated by medical professionals or therapists with the use of pelvic rehabilitation. Pelvic rehabilitation is the use of electrical stimulation to increase the muscle awareness, recruitment, strength, tone, and endurance of targeted muscles in the pelvic region.

In one type of pelvic rehabilitation program, patients are given six weeks therapy of anal electrical stimulation and weekly education on a prescribed exercise program to be performed daily in the patients' homes. The prescribed exercise program includes contracting the targeted muscle(s) for a first predetermined amount of time and relaxing the targeted muscle(s) for a second predetermined amount of time. This exercise (or cycle) is to be done for a predetermined number of times in a row and repeated a predetermined number of times each day.

Fifty percent of the treatment success has been found to be due to patient participation. The patient needs to identify the correct muscle(s), squeeze the muscle, hold the muscle, then relax the muscle. When patients are with the therapist, they are able to successfully achieve the exercises through constant education and monitoring. However, when patients are in between treatments while at home, they may perform the exercise improperly and not in accordance with the proper technique learned in the therapy sessions. The patients' muscle may become weak and it will become even harder for them to identify the proper muscle, thereby making it impossible to do the exercises correctly.

FIG. 1 illustrates a side view of the rectum and anus, which are sections of the lower gastrointestinal tract. Various muscles, muscle layers, and other layers of the rectum and anus are shown, e.g. the mucosa, the Levator Ani muscle, fatty tissue, the Puborectalis muscle, and the Dentate line. The internal sphincter and external sphincter of the anus are the muscles that are targeted to treat anal incontinence, a certain type of pelvic floor dysfunction.

Usually, diagnoses, and treatments, of anal incontinence are performed with a medical professional using an anorectal manometry device. Anorectal manometry devices are very expensive; they can cost upwards of $40,000. Because it is a very expensive device for a medical professional to purchase, it is even more impractical for a patient to have the anorectal manometry device in their home. In addition, Solesta® is a gel that is used to treat anal incontinence but is only prescribed by doctors after a proper diagnosis of a patient has been obtained using the expensive prior art anorectal manometry device. It would be desirable to eliminate the need to diagnose such conditions without using the expensive prior art anorectal manometry device.

Thus, a need exists to overcome the problems with the prior art systems, designs, and processes as discussed above.

### SUMMARY OF THE INVENTION

Disclosed is a pelvic region analyzer for performing at least one of therapeutic and diagnostic methods. In one exemplary embodiment, the pelvic region analyzer has a probe for insertion in a pelvic region of a patient. The pelvic region analyzer has a balloon for insertion in the pelvic region and is disposed at a distal end of the probe. A sensor is integrated with the probe and is disposed at a distal end of the probe. An electronic coupling is coupled to the sensor and provides pressure information from the sensor. An air tube is connected to the balloon. Air is added and removed from the air tube in accordance with at least one of the therapeutic and diagnostic methods according to the invention.

Although the invention is illustrated and described herein as embodied in a pelvic region analyzer and associated therapeutic and diagnostic methods, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Additional advantages and other features characteristic of the present invention will be set forth in the detailed description that follows and may be apparent from the detailed description or may be learned by practice of exemplary embodiments of the invention. Still other advantages of the invention may be realized by any of the instrumentalities, methods, or combinations particularly pointed out in the claims.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward.

### BRIEF DESCRIPTION OF THE DRAWINGS

The accompanying figures, where like reference numerals refer to identical or functionally similar elements throughout the separate views, which are not true to scale, and which, together with the detailed description below, are incorporated in and form part of the specification, serve to illustrate further various embodiments and to explain various principles and advantages all in accordance with the present disclosure. Advantages of embodiments of the present disclosure will be apparent from the following detailed description of the exemplary embodiments thereof, which description should be considered in conjunction with the accompanying drawings in which:

FIG. 1 is a cross-sectional view of an anus, rectum, and associated internal and external sphincter muscles;

FIG. 2 is an exploded perspective view of an exemplary embodiment of a probe, sensor, cover, and base of an analyzer according to the invention;

FIG. 3 is a bottom plan view of the base of FIG. 2;

FIG. 4 is a perspective view from the side of the analyzer of FIG. 1;

FIG. 5 is a block circuit diagram of an exemplary embodiment of a diagnostic device according to the invention;

FIG. 6 is a flow chart illustrating an exemplary embodiment of a method for providing personal pelvic floor dysfunction therapy according to the invention;

FIG. 7 is a perspective view from the side of another exemplary embodiment of an analyzer according to the invention;

FIG. 8 is a bottom perspective and partially exploded view of a base, a main air tube, a bifurcated joint, and a valve of the analyzer of FIG. 7;

FIG. 9 is a block circuit diagram of another exemplary embodiment of an analyzer according to the invention;

FIG. 10 is a flow chart of a method for providing rectal sensation compliance diagnostics, according to one embodiment;

FIG. 11 is a flow chart of a method for providing anal monometry diagnostics, according to one embodiment;

FIG. 12 is a flow chart of a method for providing rectal anal inhibitory reflex diagnostics, according to one embodiment;

FIG. 13 is a fragmentary, perspective view of an exemplary embodiment of an analyzer according to the invention;

FIG. 14 is a fragmentary, enlarged, cross-sectional view of the analyzer of FIG. 13;

FIG. 15 is a fragmentary, perspective view of the analyzer of FIG. 13 when ready to perform Rectal Sensation Threshold Tone and Compliance diagnostic tests;

FIG. 16 is a fragmentary, perspective view of the analyzer of FIGS. 13, 14, and 15;

FIG. 17 is a fragmentary, cross-sectional view of the analyzer of FIG. 13;

FIG. 18 is a fragmentary, perspective view of the analyzer of FIG. 13 when ready to perform a RAIR diagnostic test;

FIG. 19 is a perspective view of the analyzer of FIGS. 13, 17, and 18;

FIG. 20 is a fragmentary, cross-sectional plan view of the analyzer of FIG. 13;

FIG. 21 is a perspective view of another exemplary embodiment of the analyzer of FIG. 13;

FIG. 22 is a user interface flow diagram for an exemplary embodiment for operating the analyzer according to the invention;

FIG. 23 is a block circuit diagram of an exemplary embodiment of an electronics architecture according to the invention;

FIG. 24 is a diagrammatic illustration of a display shown on the analyzer according to the invention during a RAIR test with both normal and abnormal plots;

FIG. 25 is a top perspective view of a handheld device 2500 that provides biofeedback, according to one embodiment;

FIG. 26 is a perspective view of handheld device 2500 for use with a probe, according to one embodiment;

FIG. 27 is a block circuit diagram of an exemplary embodiment of an electronics architecture according to the invention; and

FIG. 28 is a device 2800 for providing pelvic region diagnostic information, according to one embodiment.

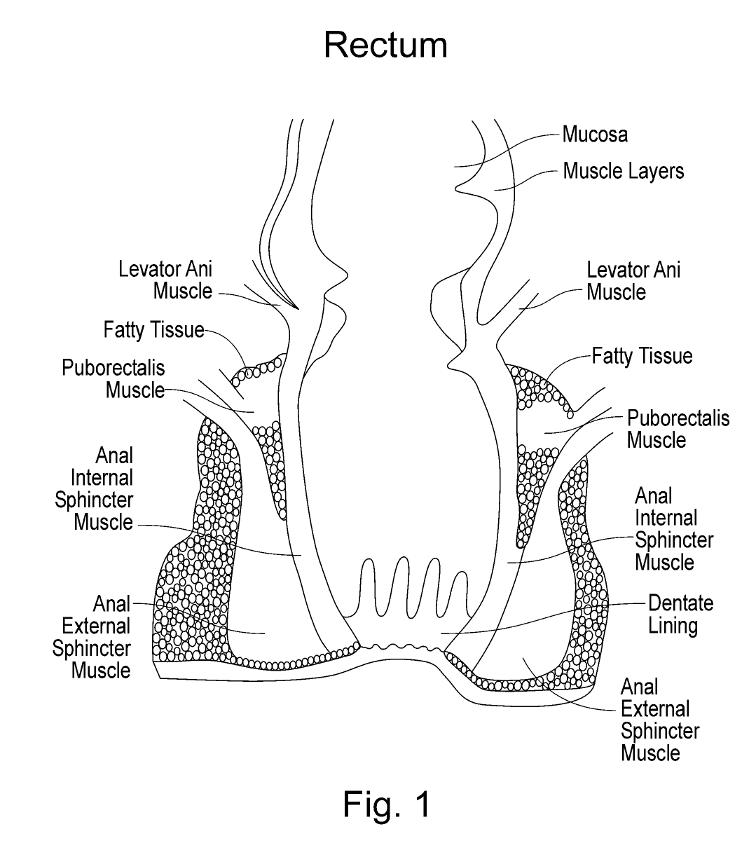

### DETAILED DESCRIPTION

As required, detailed embodiments of the present disclosure are disclosed herein; however, it is to be understood that the disclosed embodiments are merely exemplary of the disclosure, which can be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure. Further, the terms and phrases used herein are not intended to be limiting; but rather, to provide an understandable description of the disclosure. While the specification concludes with claims defining the features of the disclosure that are regarded as novel, it is believed that the disclosure will be better understood from a consideration of the following description in conjunction with the drawing figures, in which like reference numerals are carried forward.

Alternate embodiments may be devised without departing from the spirit or the scope of the disclosure. Additionally, well-known elements of exemplary embodiments of the disclosure will not be described in detail or will be omitted so as not to obscure the relevant details of the disclosure.

Before the present disclosure is disclosed and described, it is to be understood that the terminology used herein is for the purpose of describing particular embodiments only and is not intended to be limiting. The terms “a” or “an”, as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically.

Relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure.

The terms “program,” “software,” “software application,” and the like as used herein, are defined as a sequence of instructions designed for execution on a computer system. A “program,” “software,” “computer program,” or “software application” may include a subroutine, a function, a procedure, an object method, an object implementation, an executable application, an applet, a servlet, a source code, an object code, a shared library/dynamic load library and/or other sequence of instructions designed for execution on a computer system.

Herein various embodiments of the present disclosure are described. In many of the different embodiments, features are similar. Therefore, to avoid redundancy, repetitive description of these similar features may not be made in some circumstances. It shall be understood, however, that description of a first-appearing feature applies to the later described similar feature and each respective description, therefore, is to be incorporated therein without such repetition.

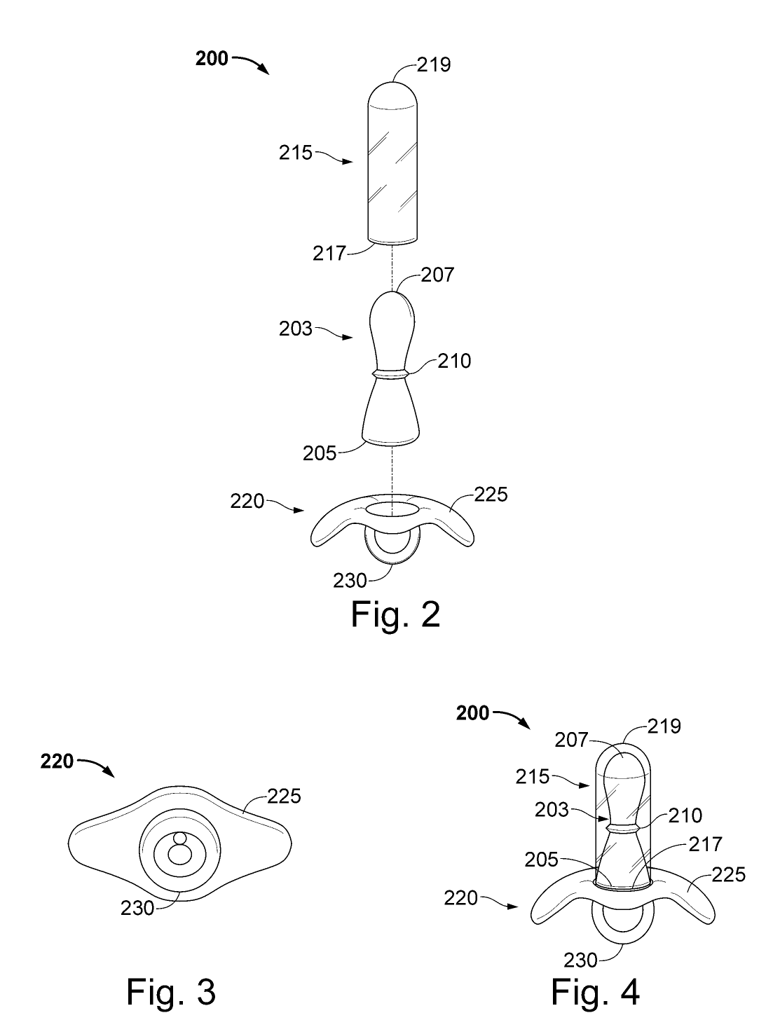

Described now are exemplary embodiments of the present disclosure. Referring now to the figures of the drawings in detail and first, particularly to FIG. 2, there is shown a first exemplary embodiment of an analyzer 200 of the present invention with the parts separated from one another. The analyzer 200 comprises a probe 203, a cover 215, and a base 220. The probe 203 has a proximal end 205 and a distal end 207. In this exemplary embodiment, the sensor 210 is disposed at an intermediate portion of probe 203, e.g., at a midpoint thereof, and is integrated with probe 203. The base 220 may be attached to the proximal end 205 of the probe 203 or, if desired, it can be fixed thereto. The base 220 has a flap 225 and a grasper 230. In an exemplary embodiment, the probe 203 is of hard plastic, such as, for example, polyethylene, polypropylene, polystyrene, polyvinyl chloride, and polytetrafluoroethylene (PTFE). The grasper 230 may be a ring, knob, or other handle that allows a user of the analyzer 200 to insert and remove the analyzer from a desired area of the user's pelvic floor region. In an exemplary embodiment, the desired pelvic floor region can be anal or vaginal. The base 220 can be of soft rubber or latex for comfort of a user.

FIG. 3 shows a view from the bottom of the base 220 with its flap 225 and grasper 230, in the form of a ring. Returning to FIG. 2, the cover 215 has a proximal end 217 and a distal end 219. The cover 215 may be placed over the probe 203 and the sensor 210. The cover 215 may be any soft material capable of keeping bodily fluids away from the sensor 210 and the probe 203. In one exemplary embodiment, cover 215 is soft rubber or latex or silicone, which allows for easier insertion and comfort as well.

FIG. 4 illustrates a side view of the exemplary embodiment of the analyzer 200. In this view, the analyzer 200 can be said to be shaped similar to a baby's pacifier. For assembly, the cover 215 is placed over the probe 203 and the sensor 210, and is sized to either snugly or loosely fit the probe 203 therein. Then, the proximal end 205 of the probe 203 is attached to the base 220 to clamp fix the cover 215 therebetween until the base 220 is removed. In the exemplary embodiment, the probe 203 and the cover 215 are fixedly attached to the base 220. Analyzer 200 may be used in conjunction with an outer covering or sleeve (similar to a thin covering used by doctors to cover thermometers so that the thermometer may be reused without having to sanitize). Analyzer 200 may also be used without such an outer sleeve, in which case it must be sanitized according to accepted medical practice and procedures.

In use for the anus, for example, the distal end 207 of the probe 203 is inserted into the rectum. The sensor 210 receives indication of a muscle squeeze, i.e., squeezing of the internal and/or external sphincter as the rectum tightens on the distal end 207. The analyzer 200 emits a sound that indicates strength of the muscle squeeze. In one exemplary embodiment, a volume of the sound increases in accordance with the strength, e.g., pressure, that is exerted by the muscle(s) on the sensor 210. Alternatively, or additionally, a frequency of the sound is changed (upwards or downwards) dependent upon the amount of pressure exerted upon the probe 203. A duration of the sound, i.e., the time that the pressure is imparted corresponding to a contraction of the muscle, indicates endurance of the exercise. Muscle squeezes by a user having a strength and endurance meeting a threshold are indicated by the analyzer 200 to the user to be “successful.” Success may be indicated to the user audibly, visually, or via the use of a vibrator.

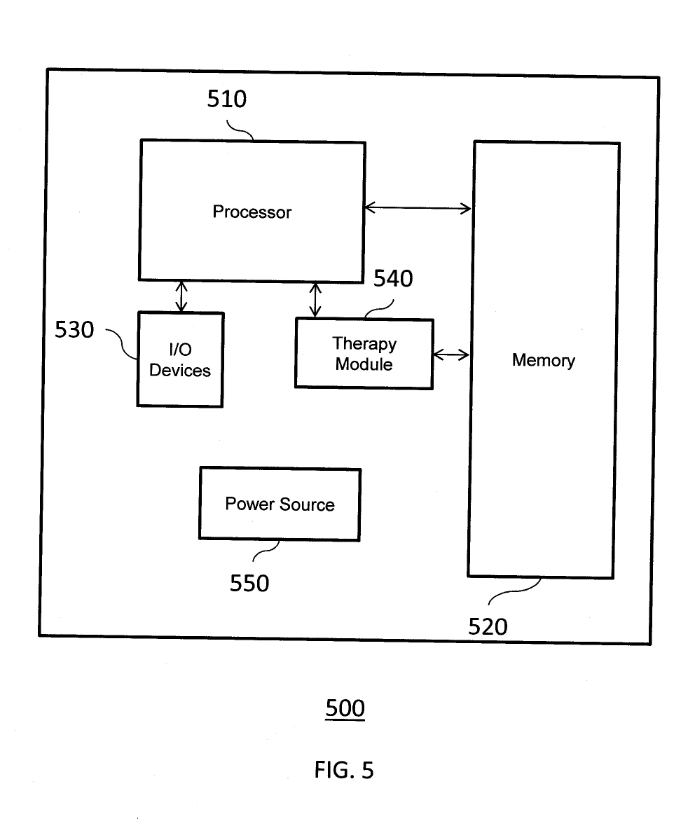

FIG. 5 is a block circuit diagram of an example device 500 for performing diagnostic tests according to the invention. Specifically, the device 500 can be employed to provide personal pelvic floor rehabilitation therapy and, therefore, may be implemented in the analyzer 200. The device 500 comprises a processor (CPU) 510, a memory 520 (e.g., random access memory (RAM) and/or read only memory (ROM)), a therapy module 540, a power source 550, and various input/output devices 530 (for example, a sensor (e.g., sensor 210), a light source operating as a visual indicator, and an indicator operating using vibration). The power source 550 may be alternating current (AC) or a battery. In one embodiment, the analyzer 200, 500 is a portable, handheld device having a rechargeable power source 550.

It should be understood that therapy module 540 can be implemented as one or more physical devices that are coupled to the CPU 510 through a communication channel. Alternatively, therapy module 540 can be represented by one or more software applications (or even a combination of software and hardware, e.g., using application specific integrated circuits (ASIC)), where the software is loaded from a storage medium, (e.g., a magnetic or optical drive or diskette) and operated by the CPU in the memory 520 of the computer. As such, therapy module 540 (including associated data structures) of the present disclosure can be stored on a non-transitory computer readable medium, e.g., RAM memory, magnetic or optical drive or diskette and the like.

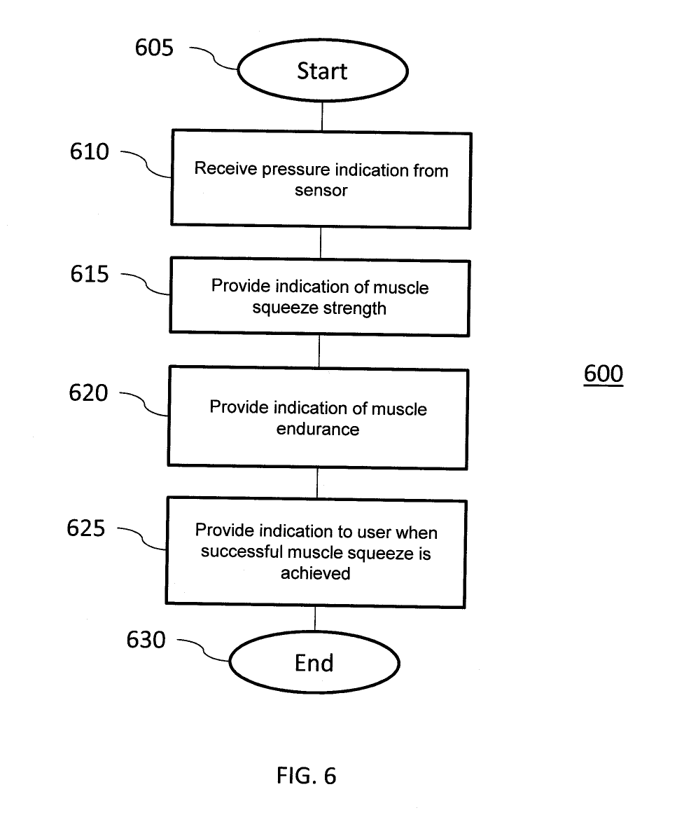

FIG. 6 illustrates a method 600 for providing personal pelvic floor dysfunction therapy according to one exemplary embodiment of the invention. Method 600 may be implemented in analyzer 200, 500. Method 600 starts at step 605 and proceeds to step 610, where a pressure indication is received from a sensor, e.g. sensor 210. At step 615, an indication of muscle squeeze strength is provided. In one embodiment, the indication of muscle squeeze strength is any or both of a volume or a frequency of a sound. For example, the volume of the sound increases in accordance with the strength of the muscle squeeze. At step 620, an indication of muscle endurance is provided. In one exemplary embodiment, the indication of muscle endurance is a length of time that the sound lasts, corresponding to a held muscle squeeze exerting a certain pressure. At step 625, an indication is provided when a successful muscle squeeze is achieved. The successful muscle squeeze indication may be provided aurally, visually, or through a vibrator indicator. Method 600 ends at step 630.

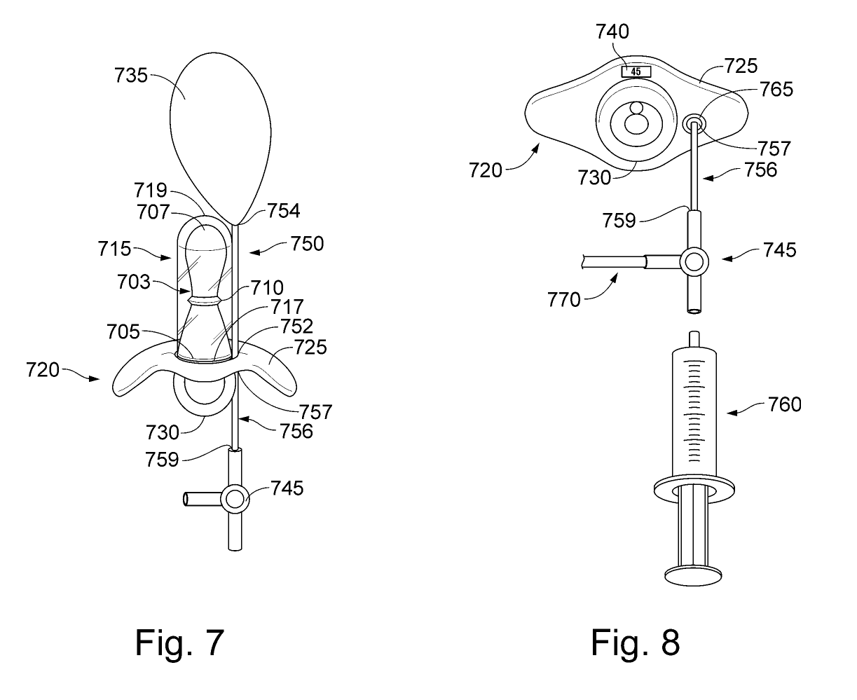

FIG. 7 illustrates another exemplary embodiment of an analyzer 700 of the present invention, which comprises a probe 703, a cover 715, a base 720, a balloon 735, a pressure reading window 740, a valve 745, a main air tube 756, and a secondary air tube 750. The probe 703 has a proximal end 705 and a distal end 707. This embodiment can serve as a device to perform a hand-held, anal/rectal manometry/rectal sensation tone and compliance test. As is known, anal/rectal manometry measures the pressure of the anal sphincter for constipation and/or anal incontinence due to carious disorders. This device is used, for example, as a diagnostic tool for patients with such constipation and/or anal incontinence.

In this exemplary embodiment, the sensor 710 is disposed at an intermediate portion of probe 203, e.g., at a midpoint thereof, and is integrated with the probe 703. The base 720 may be attached to the proximal end 705 of the probe 703 or, if desired, it can be fixed thereto. The base 720 has a flap 725 and a grasper 730. In an exemplary embodiment, the probe 203 is of hard plastic, such as, for example, polyethylene, polypropylene, polystyrene, polyvinyl chloride, and polytetrafluoroethylene (PTFE). The grasper 730 may be a ring, knob, or other handle that allows a user of the analyzer 700 to insert and remove the analyzer from a desired area of the user's pelvic floor region. In an exemplary embodiment, the desired pelvic floor region can be anal or vaginal. The base 720 can be of soft rubber or latex or silicone for comfort of a user.

The cover 715 has a proximal end 717 and a distal end 719. The cover 715 may be placed over probe 703 and sensor 710. The cover 715 may be any soft material capable of keeping bodily fluids away from the sensor 710 and the probe 703. In one exemplary embodiment, the cover 715 is soft rubber or latex, which allows for easier insertion and comfort as well.

The analyzer 700 can be said to be shaped similar to a baby's pacifier. For assembly, the cover 715 is places over the probe 703 and the sensor 710, and is sized to either snugly or loosely fit the probe 703 therein. Then, the proximal end 705 of the probe 703 is attached to base 720 to clamp fix the cover 715 therebetween until the base 720 is removed.

Also provided is a balloon assembly. The secondary air tube 750 of the balloon assembly has a proximal end 752 and a distal end 754. The secondary air tube 750 is placed alongside the cover 715 and runs to a bifurcated joint 765 (see FIG. 8) at flap 725 of base 720. The distal end 754 of the secondary air tube 750 is attached to the balloon 735. In one exemplary embodiment, the balloon 735, when inflated is extended six inches above the distal end 719 of the cover 715. In one exemplary embodiment, the probe 703 and the cover 715 are fixedly attached to base 720. Analyzer 700 may be used in conjunction with an outer covering or sleeve (similar to a thin covering used by doctors to cover thermometers so that the thermometer may be reused without having to sanitize). Analyzer 700 may also be used without such an outer covering, in which case, it must be sanitized according to accepted medical practices and procedures.

Viewed from the bottom of the base 720, FIG. 8 shows a main air tube 756, a bifurcated joint 765, and a valve 745. The proximal end 752 (see FIG. 7) of the secondary air tube 750 is attached to the bifurcated joint 765. A distal end 757 of the main air tube 756 is attached to the bifurcated joint 765 at the flap 725. A proximal end 759 of the main air tube 756 is attached to the valve 745, which is used to prevent air from expelling out from balloon 735 until desired. In one exemplary embodiment, the valve 745 is a luer-lock, two-way valve with the main air tube 756 attached to the valve 745. In one exemplary embodiment, the valve 745 may have a sensor 770 that measures pressure on the balloon 735. In another exemplary embodiment, the valve 745 may also be attached to a tube 770 (which is coupled to an external device) that is used to measure pressure on balloon 735.

The base 720 includes a pressure reading window 740, which may be integrated in the flap 725 or the grasper 730, for example, in a case where the grasper 730 is a knob or any other solidly shaped object allowing for placement of the pressure reading window. In one exemplary embodiment, the pressure reading window 740 is a digital pressure reading window. The pressure reading window 740 is electronically coupled (coupling not shown) to sensor 710 and provides pressure information from the sensor 710 to the pressure reading window 740.

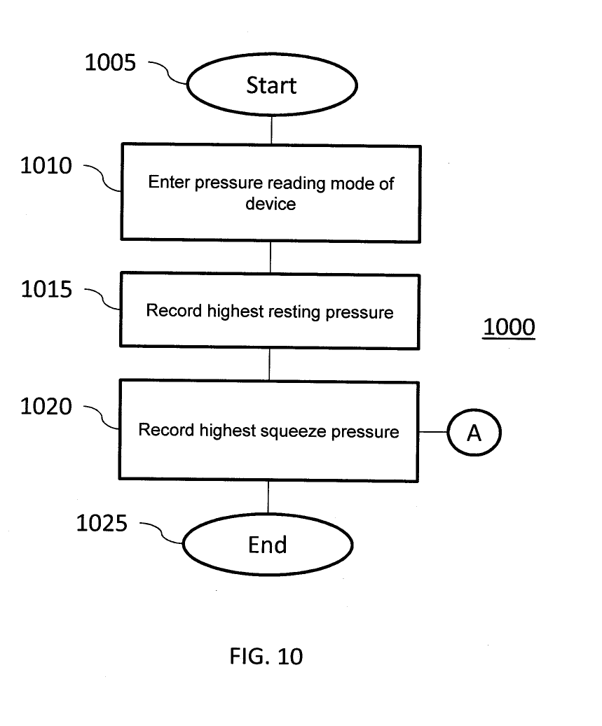

During an exemplary diagnostic test according to the invention, the analyzer 700, including the balloon 735, is inserted into the pelvic floor region of the patient. In one exemplary embodiment, a distal end of the analyzer 700 (e.g., distal ends 707, 719) is inserted into the rectum of a patient until the flap 725 of the analyzer 700 abuts the anus of the patient. Air is caused to enter the balloon 735 (e.g., using a syringe 760) through the valve 745. The pressurized air fills the balloon 735 through the main 756 and secondary 750 air tubes. Diagnostic tests (as described below with respect to FIGS. 10, 11, and 12) may be performed on the patient by a medical professional using responses from the patient and readings from pressure reading window 740. These tests may be performed when the balloon 735 is empty, as the balloon 735 fills with air, or as air is released from the balloon 735, as set forth in accordance with the diagnostic tests.

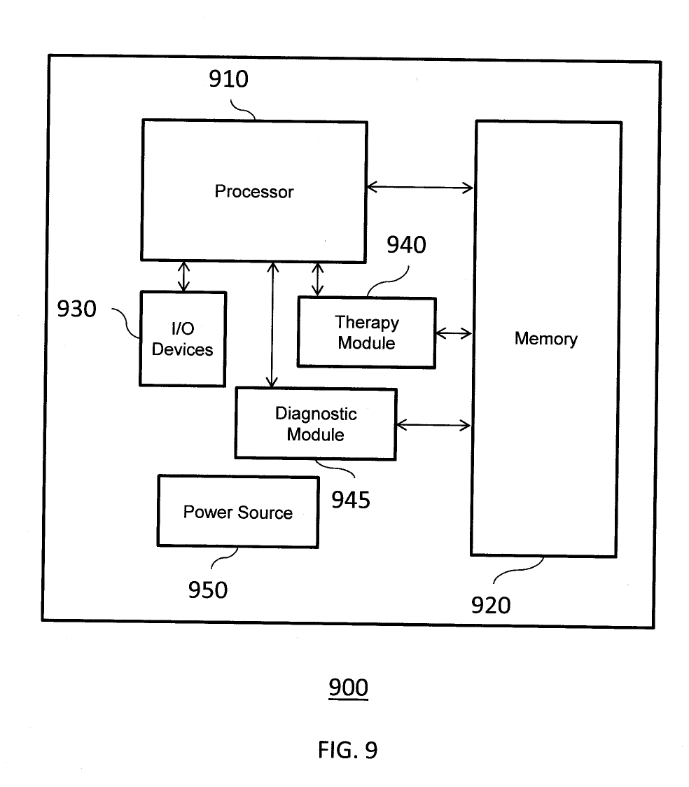

FIG. 9 is a block circuit diagram of an example device 900 for performing diagnostic tests according to the invention. Specifically, the device 900 can be employed to provide pelvic floor rehabilitation therapy and diagnostics and, therefore, may be implemented in the analyzer 700. The device 900 comprises a processor (CPU) 910, a memory 920 (e.g., a random access memory (RAM) and/or read only memory (ROM)), a therapy module 940, a diagnostic module 945, a power source 950, and various input/output devices 930 including, for example, a sensor (e.g., sensor 710), a light source operating as a visual indicator, an indicator operating using vibration, storage devices, including but not limited to, a tape drive, a floppy drive, a hard disk drive or a compact disk drive, a receiver, a transmitter, a Universal Serial Bus (USB) mass storage, a network attached storage, and/or a storage device on a network cloud).

A receiver 930 and a transmitter 930 (e.g., transceiver) may be used for short-range wireless protocol communication, e.g. Wi-Fi, Bluetooth. The device 700, 900 may communicate with a smart phone, a tablet, or another computing device via the short-range wireless protocol in order to provide data from the device 700, 900 to a diagnostic application residing on the smart phone, tablet, or computing device. Likewise, the device 700, 900 may communicate with a smart phone, tablet, or other computing device using a USB connection. Data may be provided from device 700, 900 to the diagnostic application residing on the smart phone, tablet, or other computing device using the USB connection. The power source 950 may be alternating current (AC) or a battery. In one exemplary embodiment, the analyzer 700, 900 is a portable, handheld device having a rechargeable power source 950.

It should be understood that therapy module 940 and diagnostic module 945 can be implemented as one or more physical devices that are coupled to the CPU 910 through a communication channel. Alternatively, therapy module 840 and diagnostic module 945 can be represented by one or more software applications (or even a combination of software and hardware, e.g., using application specific integrated circuits (ASIC)), where the software is loaded from a storage medium, (e.g., a magnetic or optical drive or diskette) and operated by the CPU in the memory 920 of the computer. As such, therapy module 940 and diagnostic module 945 (including associated data structures) of the present disclosure can be stored on a non-transitory computer readable medium, e.g., RAM memory, magnetic or optical drive or diskette and the like.

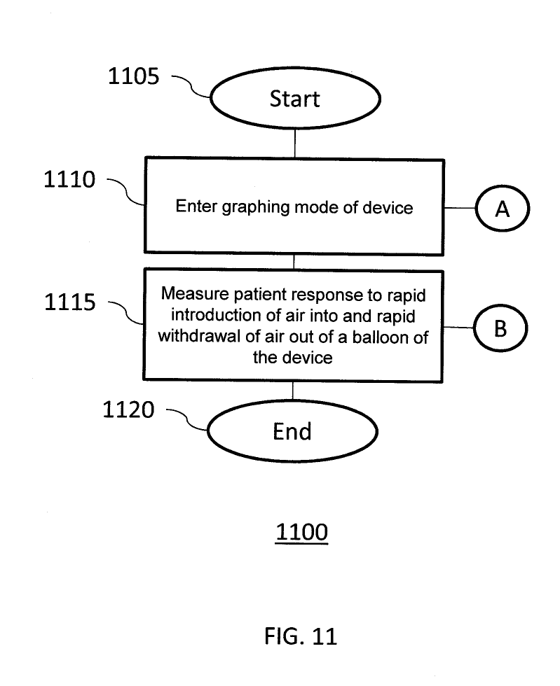

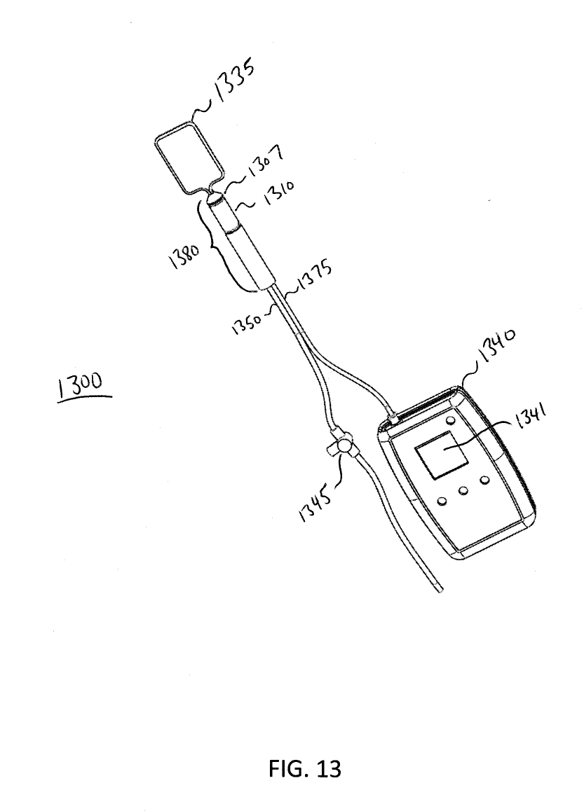

FIG. 10 illustrates a diagram of a method 1000 according to one exemplary embodiment of the invention. Method 1000 comprises an anal manometry diagnostic test for performance by a device, e.g., device 700, 900. Method 1000 begins at step 1005. A patient is placed in a left lateral position. Spine alignment is not needed when using the analyzer 700, 900. The analyzer 700, 900 is lubricated and inserted into the rectum to a 6 cm marking of secondary tube 750 at the anal verge. As a frame of reference, the balloon 735 is located at a 1 cm marking on the secondary tube 750. While looking at the analyzer display screen 740 (whether on the base 220, 720 or on a separate, device communicating with the sensor 210, 710, for example, in FIGS. 13, 16, 19, and 21), a diagnostician using the device slowly moves the analyzer back and forth from the 6 cm mark to a 2 cm marker at the anal verge to find and record both the resting and highest pressure. Typically, the highest resting pressure is found when the marker of secondary tube 750 is between 3 cm and 4 cm inferior of the anal verge. At step 1010, a pressure reading mode of the device is entered. Pressure readings may be read from display screen 740, whether on the base 220, 720 or on the separate device. The patient rests quietly with no squeezing or straining for 20-30 seconds while a resting pressure is recorded at step 1015. Resting pressure approximates the internal anal sphincter muscle. At step 1020, a highest squeeze pressure is recorded. The patient is instructed to squeeze as hard as possible for 5 seconds. The maximum squeeze pressure is then recorded. Maximum squeeze pressure approximates the external anal sphincter muscle. Method 1000 either ends at step 1025, or proceeds to step 1110 of FIG. 11.

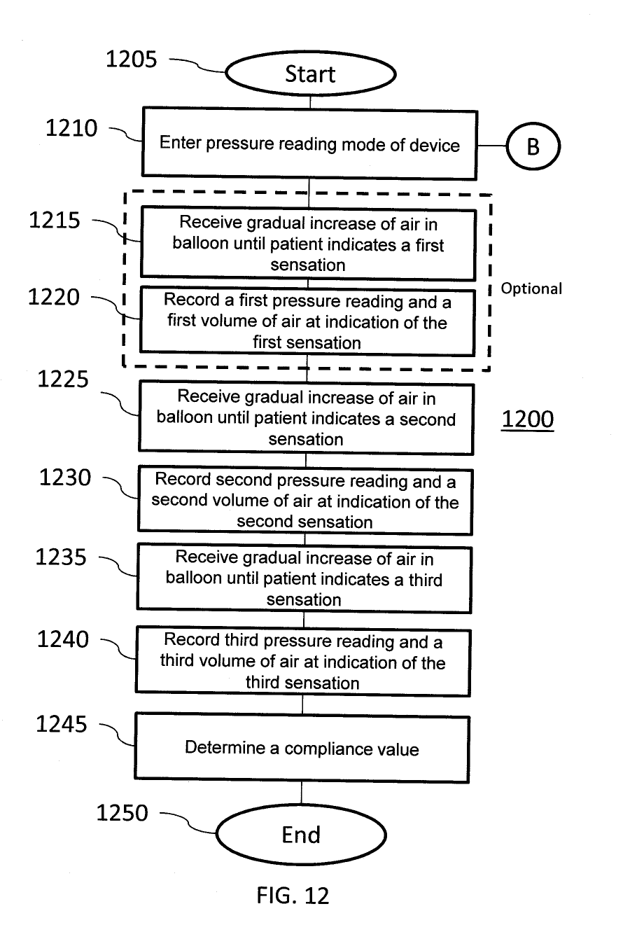

FIG. 11 illustrates a diagram of a method 1100 according to another exemplary embodiment of the invention. Method 1100 comprises a Recto-Anal Inhibitory Reflex (RAIR) diagnostic test performed by device 700, 900. Method 1100 either starts at step 1105 or is initiated after a user proceeds from step 1020 to step 1110. At step 1110, a graphing mode of the device is entered. Device 700, 900 should be within the rectum at the high resting pressure zone of the patient, e.g., where the marker of secondary tube 750 is about 3 cm to 4 cm inferior of the anal verge. At step 1115, a patient response to rapid introduction of air into and rapid withdrawal of air out from the balloon 735 is measured. Using an inflation device, e.g., syringe 760, 40 cc to 60 cc of air is abruptly plunged into balloon 735 and, within 2 to 4 seconds thereafter, the air is completely withdrawn from the balloon to identify whether RAIR is present. When balloon 735 is rapidly inflated, a normal patient will exhibit a particular reflex. Absence of such a reflex is a clear indication of Hirschsprung's disease. Method 1100 either ends at step 1120, or proceeds to step 1210 of FIG. 12.

FIG. 12 illustrates a diagram of a further method 1200 according to an exemplary embodiment of the invention. Method 1200 comprises a Rectal Sensation Threshold Tone and Compliance diagnostic test performed by device 700, 900. Method 1200 either starts at step 1205 or is initiated after a user proceeds from step 1115 to step 1210. At step 1210, a pressure reading mode of the device is entered. The balloon 735 is placed in the rectal ampulla of a patient, e.g., where the marker of the secondary tube 750 is at 6 cm interior of the anal verge. The patient will be instructed to respond to at least two sensory thresholds. At step 1215, a gradual increase of air is received in the balloon 735 until the patient indicates a first sensation—the point at which a patient starts to feel the balloon 735 filling with air. With the indication of the first sensation, at step 1220, a first pressure reading and a first volume of air are recorded. At step 1225, a further gradual increase of air is received in the balloon 735 until the patient indicates a second sensation—the point at which the patient feels the need to defecate. With the indication of the second sensation, at step 1230, a second pressure reading and a second volume of air are recorded. At step 1235, a further gradual increase of air is received in the balloon 735 until the patient indicates a third sensation—the point at which the patient is at a maximum tolerable sensation to defecate (i.e., the patient feels that they can no longer hold in their feces). At the indication of the third sensation, at step 1240, a third pressure reading and a third volume of air are recorded. At step 1245, a compliance value is determined based upon the pressure readings. These pressure readings are recorded on the hand-held device 700, 900 so that the medical professional may make notes in the patient's chart. Typical ranges for first, second, and third sensation recorded air volumes are 40 cc to 90 cc, 120 cc to 140 cc, and 200 cc to 300 cc, respectively. Prior art devices only record up to 300 cc maximum air volume. However, the present device 700, 900 may allow for recording of pressure values at volumes greater than 300 cc. The compliance value may be computed according to a difference of volumes divided by a difference of the two corresponding pressure readings, e.g., (V3−V2)/(P3−P2). The compliance value may also be computed by a recorded volume by its corresponding pressure reading, e.g. V2/P2. In one embodiment, the first sensation portion of the test is optional. In this embodiment, the medical professional performing the test would increase the amount of air in the balloon 735 until the patient feels the need to defecate.

FIG. 13 illustrates a perspective view of an analyzer 1300 according to one exemplary embodiment. In this embodiment, the analyzer is set up to perform an anal manometry diagnostic procedure, e.g., the procedure described in method 1000. Analyzer 1300 comprises a balloon 1335, a probe 1380, a sensor 1310, a main air tube 1350, an electrical connection 1375, a pressure reading device 1340, and a valve 1345. The main air tube 1350 is disposed within the probe 1380 and is slideably engageable within the probe 1380. In this embodiment, the balloon 1335 is retracted to a distal end 1307 of the probe 1380 in order to perform an anal manometry diagnostic procedure, however for anal manometry it does not matter where the balloon is placed. To begin the anal manometry diagnostic procedure, a patient is placed in a left lateral position. Spine alignment is not needed when using the analyzer 1300. The analyzer 1300 is lubricated and inserted into the rectum. The analyzer pressure reading device 1340 communicates with the sensor 1310 via the electrical connection 1375. While looking at the analyzer pressure reading device display screen 1341, a diagnostician using the device 1300 slowly moves the analyzer back and forth in the patient's rectum to find and record both the resting and highest pressure. A pressure reading mode of device 1340 is entered. Pressure readings may be read from display screen 1341, for example, with a number indicating psi or inches of mercury. The patient rests quietly with no squeezing or straining for 20-30 seconds while a resting pressure is recorded. The resting pressure approximates the internal anal sphincter muscle. The patient is instructed to squeeze as hard as possible for 5 seconds. The maximum squeeze pressure is then recorded by the device 1340. Maximum squeeze pressure approximates the external anal sphincter muscle.

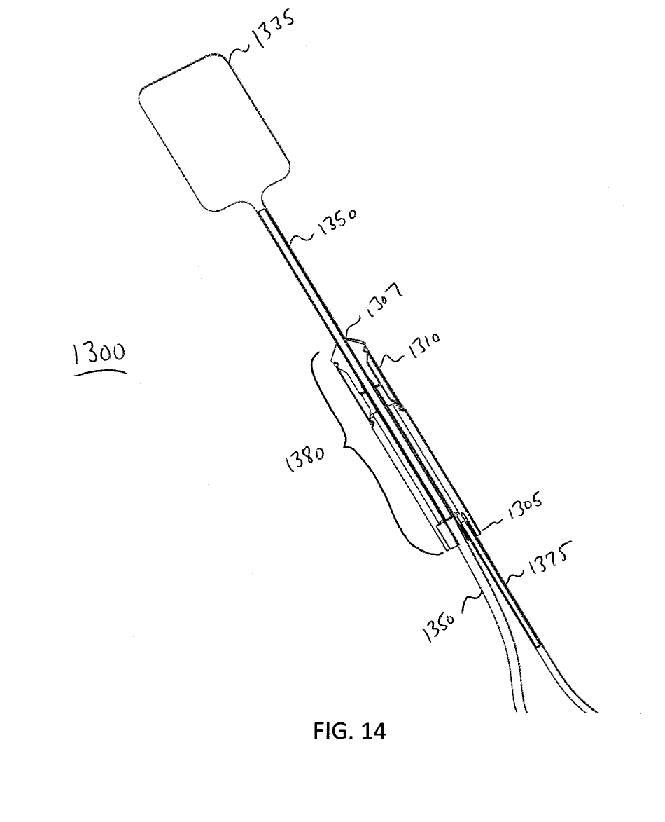

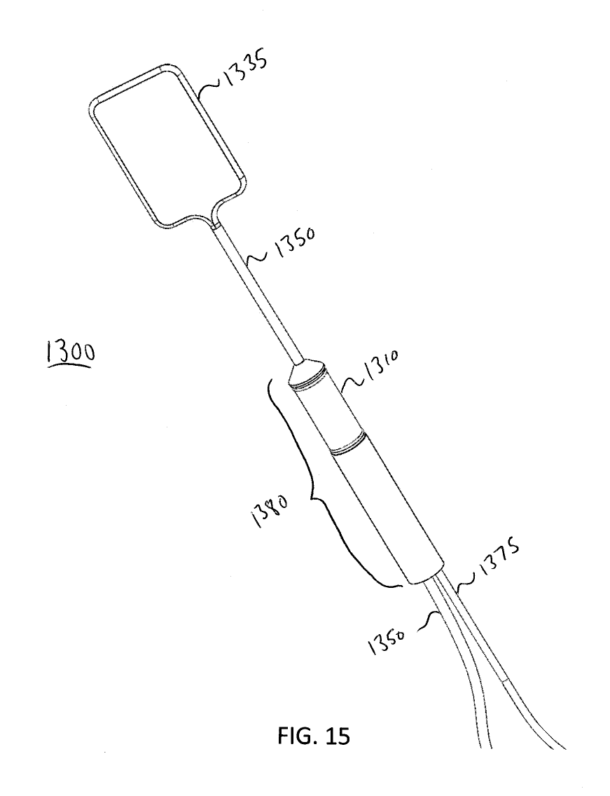

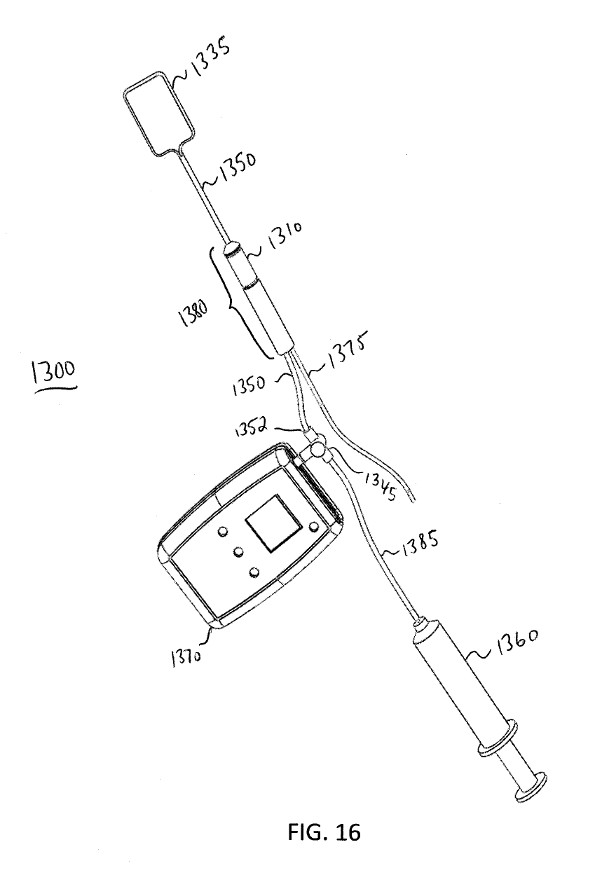

FIG. 14 illustrates a cross-sectional view of a distal end of the analyzer 1300. In this exemplary embodiment, the analyzer 1300 is set up to perform a Rectal Sensation Threshold Tone and Compliance diagnostic test. The analyzer 1300 comprises the balloon 1335, the probe 1380, the sensor 1310, the main air tube 1350, and the electrical connection or coupling 1375. In this embodiment, the balloon 1335 is extended from a distal end 1307 of probe 1380 in order to perform a Rectal Sensation Threshold Tone and Compliance diagnostic procedure, e.g., the procedure described in method 1200. The main air tube 1350 may be pushed through an opening in the proximal end 1305 of the probe 1380 in order to extend the balloon 1335 from the interior of the distal end 1307 of probe 1380, and further into a patient's rectum in order to perform the procedure. FIG. 15 illustrates a perspective view of the analyzer 1300 when the analyzer 1300 is set up to perform the Rectal Sensation Threshold Tone and Compliance diagnostic test. FIG. 16 illustrates a further embodiment of the elements shown in FIGS. 14 and 15. In this embodiment, FIG. 16 additionally shows a valve 1345, a pressure reading device 1370, an air tube 1385, and a syringe 1360. The proximal end 1352 of the air tube 1350 is attached to a distal side of the valve 1345 (with respect to the syringe 1360). The valve 1345 is used to prevent air from expelling out from balloon 1335 until desired. In one exemplary embodiment, the valve 1345 is a luer-lock, two-way valve. The valve 1345 is coupled to a pressure reading device 1370 that measures pressure on the balloon 1335.

When the balloon 1335 is extended as shown in FIGS. 14, 15, and 16, analyzer 1300 may perform a Rectal Sensation Threshold Tone and Compliance diagnostic test, e.g., method 1200. A pressure reading mode of the device 1370 is entered. The balloon 1335 is extended distally from a distal end 1307 of probe 1380 such that the balloon 1335 is placed in the rectal ampulla of a patient. The patient will be instructed to respond to at least two sensory thresholds. A gradual increase of air is received in the balloon 1335 until the patient indicates a first sensation—the point at which a patient starts to feel the balloon 1335 filling with air. With the indication of the first sensation a first pressure reading and a first volume of air are recorded. A further gradual increase of air is received in the balloon 1335 until the patient indicates a second sensation—the point at which the patient feels the need to defecate. With the indication of the second sensation, a second pressure reading and a second volume of air are recorded. A further gradual increase of air is received in the balloon 1335 until the patient indicates a third sensation—the point at which the patient is at a maximum tolerable sensation to defecate (i.e., the patient feels that they can no longer hold in their feces). At the indication of the third sensation, a third pressure reading and a third volume of air are recorded. A compliance value is determined based upon the pressure readings. These pressure readings are recorded on the hand-held device 1370 so that the medical professional may make notes in the patient's chart. Typical ranges for first, second, and third sensation recorded air volumes are 40 cc to 90 cc, 120 cc to 140 cc, and 200 cc to 300 cc, respectively. Prior art devices only record up to 300 cc maximum air volume. However, the device of the present invention may allow for recording of pressure values at volumes greater than 300 cc. The compliance value may be computed according to a difference of volumes divided by a difference of the two corresponding pressure readings, e.g., (V3−V2)/(P3−P2). The compliance value may also be computed by a recorded volume through its corresponding pressure reading, e.g., V2/P2. In one exemplary embodiment, the first sensation portion of the test is optional. In this embodiment, the medical professional performing the test would increase the amount of air in the balloon 1335 until the patient feels the need to defecate.

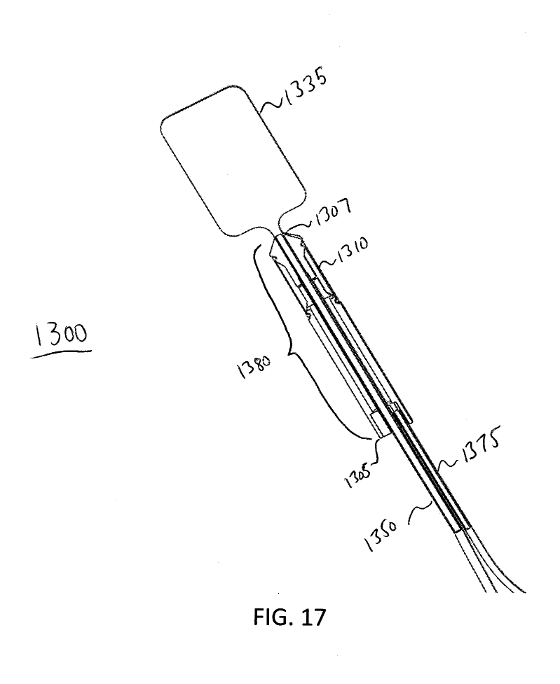

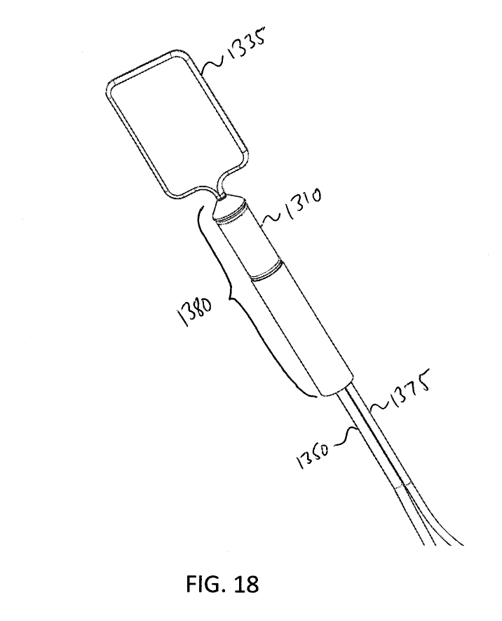

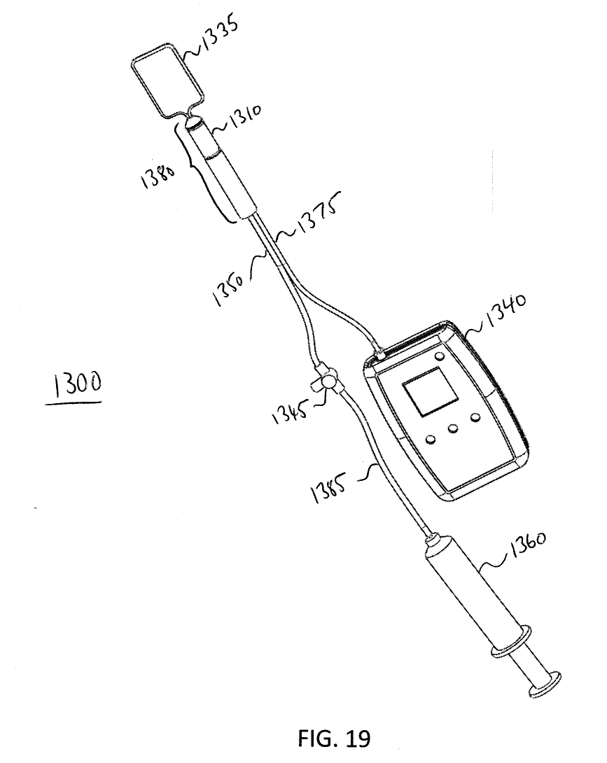

FIG. 17 illustrates a cross-sectional view of the analyzer 1300. In this exemplary embodiment, the analyzer is set up to perform a Recto-Anal Inhibitory Reflex (RAIR) diagnostic test. Analyzer 1300 comprises the balloon 1335, the probe 1380, the sensor 1310, the main air tube 1350, and an electrical connection 1375. In this embodiment, the balloon 1335 is retracted to a distal end 1307 of probe 1380 in order to perform a RAIR diagnostic procedure, e.g. the procedure described in method 1100. Air tube 1350 may be pulled through an opening in the proximal end 1305 of probe 1380 in order to retract balloon 1335 to the distal end 1307 of probe 1380 in order to perform the procedure. FIG. 18 illustrates a perspective view of analyzer 1300 when the analyzer is set up to perform the RAIR diagnostic test. FIG. 19 illustrates a further embodiment of the elements shown in FIGS. 17 and 18. In this embodiment, FIG. 19 additionally shows a valve 1345, a pressure reading device 1340, an air tube 1385, and a syringe 1360. The proximal end 1352 of the air tube 1350 is attached to the distal side of the valve 1345. The valve 1345 is used to prevent air from expelling out from balloon 1335 until desired. In one exemplary embodiment, the valve 1345 is a luer-lock, two-way valve. The valve 1345 is coupled to a pressure reading device 1340 that measures pressure from the sensor 1310.

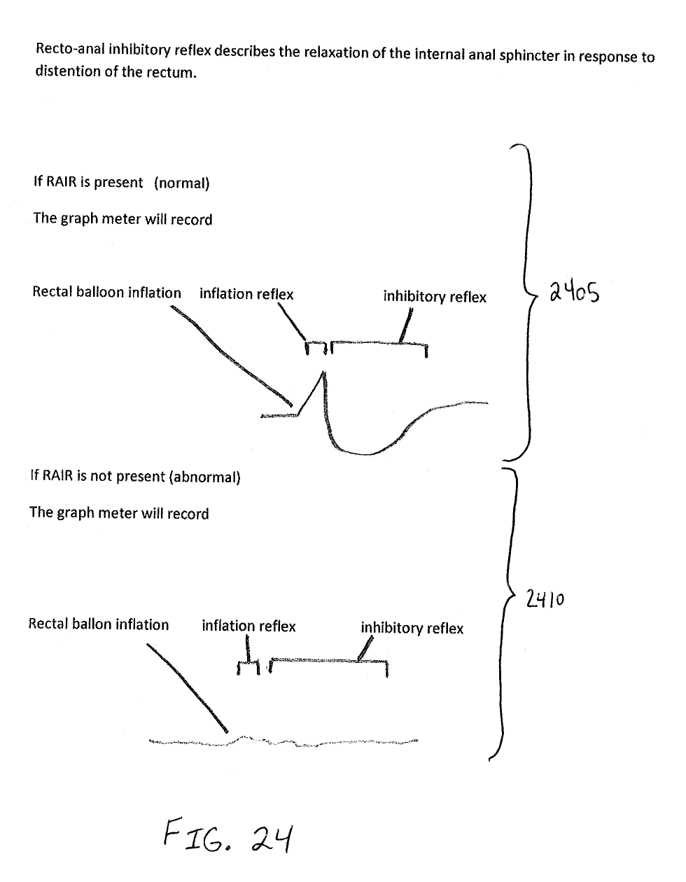

The analyzer 1300 may perform a Recto-Anal Inhibitory Reflex (RAIR) diagnostic test, e.g., in accordance with method 1100. A graphing mode of the device 1340 is entered. The analyzer 1300 should be within the rectum at the high resting pressure zone of the patient. A patient response to rapid introduction of air into and rapid withdrawal of air out from the balloon 1335 is measured. The balloon is inserted further into the rectum and, using an inflation device, e.g., syringe 1360, 40 cc to 60 cc of air is abruptly plunged into balloon 1335. Within 2 to 4 seconds thereafter, the air is completely withdrawn from the balloon. The pressure reading on the display 1341 can be used to identify whether RAIR is present. Exemplary graphs 2405, 2410 in FIG. 24 showing possible visual responses for a RAIR diagnostic test on a graph meter, e.g., a graph mode presented on display 1341, for both normal and abnormal conditions. Recto-anal inhibitory reflex describes the relaxation of the internal anal sphincter in response to distention of the rectum. Graph 2405 shows a visual representation of what a graph meter would record if RAIR is present, i.e., a normal condition. Graph 2410 shows a visual representation of what a graph meter would record if RAIR is not present, i.e., an abnormal condition. When the balloon 1335 is rapidly inflated, a normal patient will exhibit a particular reflex shown, for example, in graph 2405. Absence of such a reflex (see graph 2410) is a clear indication of Hirschsprung's disease.

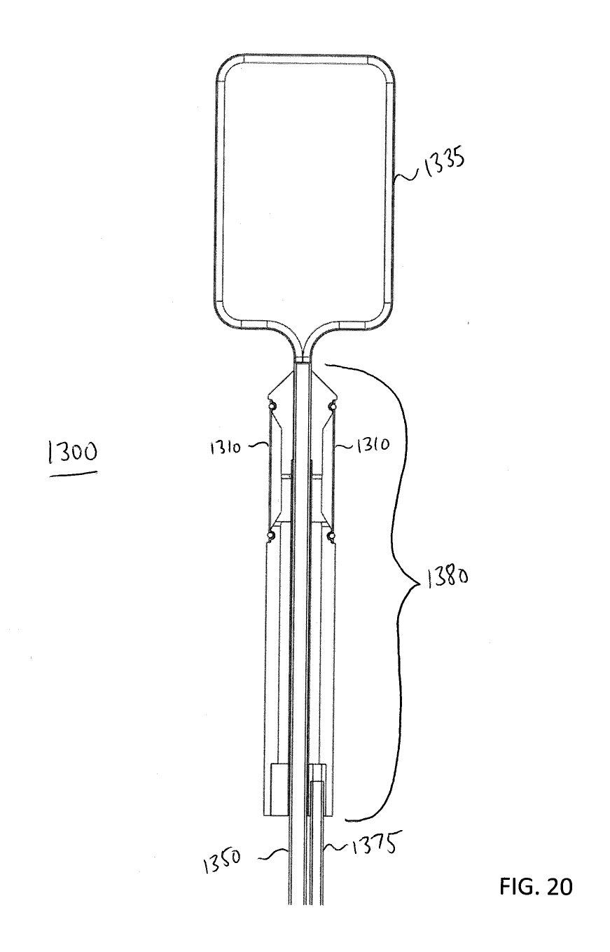

FIG. 20 illustrates a cross-sectional view of the analyzer 1300. In this exemplary embodiment, the analyzer 1300 is set up to perform either an anal manometry diagnostic test or a Recto-Anal Inhibitory Reflex (RAIR) diagnostic test. The analyzer 1300 comprises the balloon 1335, the probe 1380, the sensor 1310, the main air tube 1350, and the electrical connection 1375. In this embodiment, the balloon 1335 is extended from a distal end 1307 of probe 1380 in order to perform, e.g., methods 1000 and 1100. The air tube 1350 may be pulled through an opening in the proximal end 1305 of probe 1380 in order to retract balloon 1335 to the distal end 1307 of probe 1380 in order to perform any of the diagnostic procedures as desired.

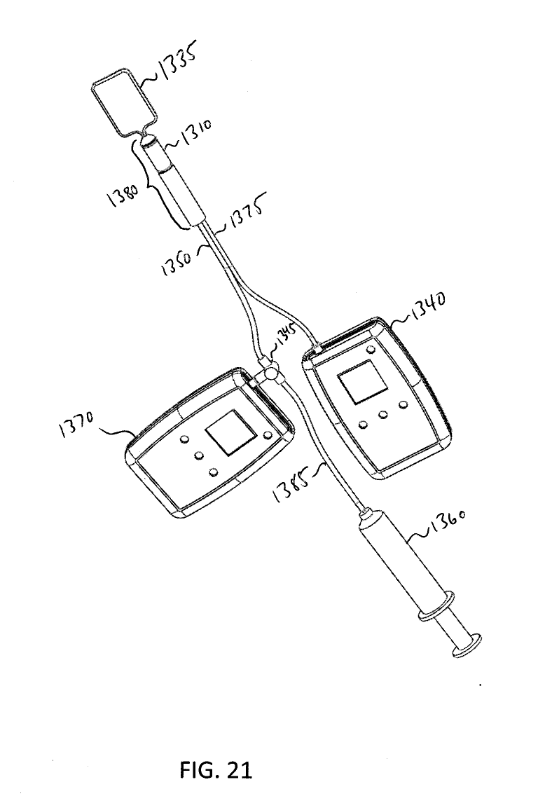

FIG. 21 illustrates a perspective view of the analyzer 1300, which comprises the balloon 1335, the probe 1380, the sensor 1310, the main air tube 1350, and the electrical connection 1375. In this exemplary embodiment, the balloon 1335 is extended from a distal end 1307 of the probe 1380 in order to perform, e.g., methods 1000 and 1100. The air tube 1350 may be pulled through an opening in the proximal end 1305 of probe 1380 in order to retract balloon 1335 to the distal end 1307 of probe 1380 and perform any of the diagnostic procedures mentioned. In this embodiment, additionally shown is a valve 1345, a pressure reading device 1370, an air tube 1385, and syringe 1360. The proximal end 1352 of air tube 1350 is attached to the distal side of the valve 1345. The valve 1345 is used to prevent air from expelling out from balloon 1335 until desired. In one exemplary embodiment, the valve 1345 is a luer-lock, two-way valve. The valve 1345 is coupled to a pressure reading device 1370 that measures pressure on the balloon 1335. The valve 1345 is also coupled to a pressure reading device 1340 that measures pressure from sensor 1310. Although pressure reading devices 1340, 1370 are shown as separate devices, the functionality for each of these devices may be combined into one pressure reading device that measures pressures from sensor 1310 and pressures exerted on balloon 1335.

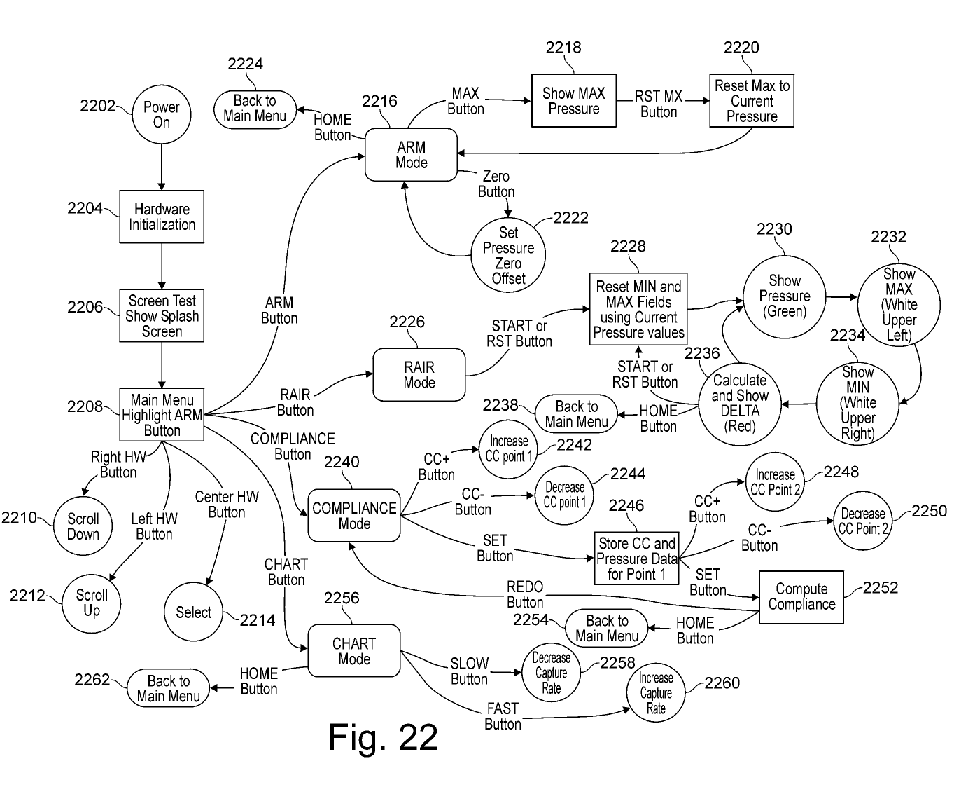

FIG. 22 illustrates a user interface flow diagram for the analyzer 700, 900, 1300 according to one exemplary embodiment of the invention. The analyzer is powered on at step 2202. At step 2204, hardware initialization is performed. At step 2206, a screen test is performed and, for example, a splash screen is shown. At step 2208, the main menu is shown. In one exemplary embodiment, an Ano-Rectal Manometry (ARM) button is highlighted initially. Although the present embodiments show a device having a device that uses a screen in conjunction with hardware buttons, the present disclosure also contemplates use of a touch screen and soft keys or any other equivalent user interfaces, including voice commands. A user of the device 700, 900, 1300 may use one hardware button to scroll down 2210 (e.g., a right button), another hardware button to scroll up 2212 (e.g., a left button), and a third hardware button 2214 (e.g., a center button) to select a diagnostic test.

When the ARM mode option is selected, ARM mode is entered at 2216. A current maximum pressure is shown 2218 when a MAX button is selected. The MAX may be reset to a current pressure 2220 by pressing a RST MX button. A zero offset of a pressure may be set at 2222 by pressing a ZERO button. A user may return to the main menu 2224 by pressing a HOME button.

When the RAIR mode option is selected, RAIR mode is entered at 2226. At step 2228, MIN and MAX fields may be reset using current pressure values when a user presses the START or RST button. A pressure is shown 2230 and may be illustrated as green, for example. A MAX pressure is shown 2232 and may be illustrated in white in an upper left section of the device screen, for example. A MIN pressure is shown 2234 and may be illustrated in white in an upper right section of the device screen, for example. A difference (DELTA) between MAX 2232 and MIN 2234 is calculated at 2236 and shown on the device screen. DELTA is signified, for example, by the color red. A user may redo the diagnostic test by pressing the START or RST button. The user may return to the Main Menu 2238 by pressing the HOME button.

When a COMPLIANCE button is selected, the COMPLIANCE mode is entered at 2240. At least two data points are captured during a COMPLIANCE test. In one embodiment, three data points may be captured. A CC+ button may be used to increase air volume in the balloon, e.g., balloon 735, 1335, to obtain a first data point 2242. A CC− button is used in order to decrease air volume in the balloon for the first data point 2244. At step 2246, volume (CC) and pressure data are stored for the first data point. A CC+ button is used in order to increase air volume in the balloon for a second data point 2248. A CC− button is used in order to decrease air volume in the balloon for the second data point 2250. At step 2252, a compliance value is computed when a user selects the SET button. A user may redo the diagnostic test by pressing the REDO button and may return to the Main Menu 2254 by pressing the HOME button.

When a CHART button is selected, a CHART mode is entered at 2256. A capture rate is decreased at step 2258 by pressing the SLOW button. A capture rate is increased at step 2260 by pressing a FAST button. A user may return to the Main Menu 2262 by pressing the HOME button.

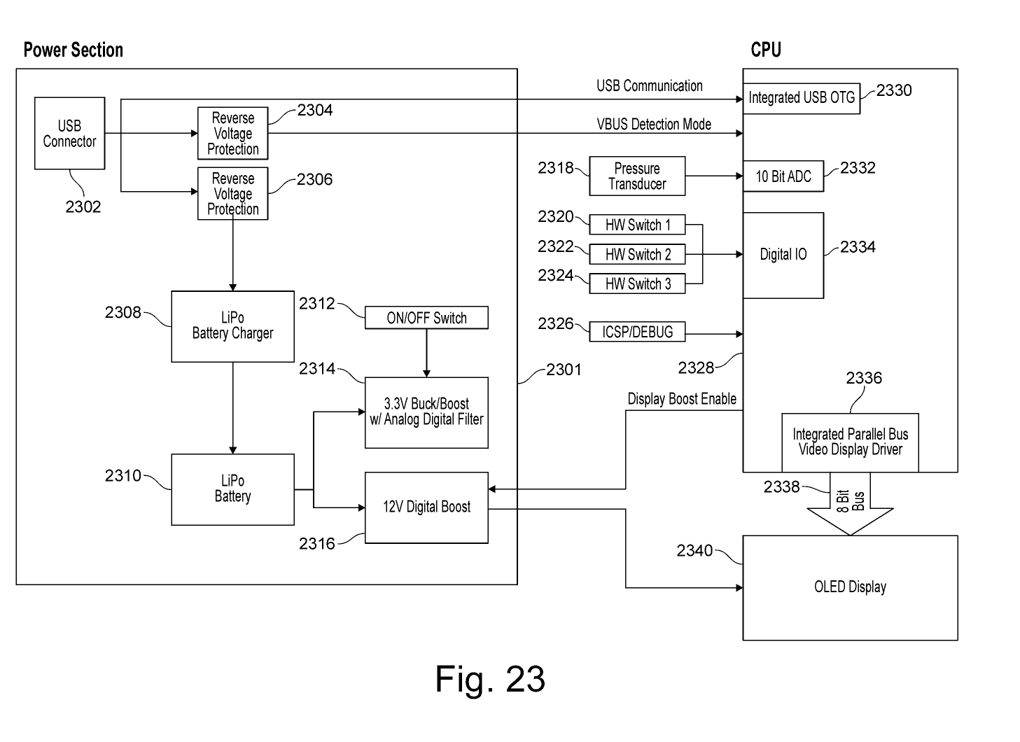

FIG. 23 illustrates an electronics architecture 2300 according to one exemplary embodiment of the invention. Electronics architecture 2300 comprises a power section 2301, a central processing unit (CPU) section 2328, and a display 2340. Power section 2301 has a universal serial bus (USB) connector 2302 that is coupled to communicate with a CPU 2328 through an integrated USB on-the-go (OTG) interface 2330. The USB connector 2302 communicates virtual bus (VBUS) Detection Mode information to CPU 2328 through reverse voltage protection 2304. In addition, the USB connecter 2302 provides power to the battery charger 2308 through the reverse voltage protection 2306. Battery charger 2308 charges the battery 2310. In one exemplary embodiment, the battery charger 2308 is a Lithium Polymer (LiPo) battery charger and the battery 2310 is a LiPo battery. The battery 2310 is coupled to buck/boost power supply 2314 and a display boost 2316. In one exemplary embodiment, the buck/boost 2314 is a 3.3V Buck/Boost with an Analog/Digital filter. In one exemplary embodiment, the display boost 2316 is a 12V display boost. The power section 2301 also includes an ON/OFF switch 2312 coupled to the buck/boost 2314.

The CPU section 2328 is connected to a pressure transducer 2318 through a 10-bit analog-to-digital converter 2332. The CPU 2328 is also connected with hardware switches 2320, 2322, 2324 through a digital input/output 2334. The CPU 2328 sends information to the display 2340 through an integrated parallel Bus video display driver 2336 and an 8-bit Bus 2338. In one exemplary embodiment, display 2340 is an OLED display.

FIG. 24 illustrates graphs 2405, 2410 showing possible visual responses for a RAIR diagnostic test on a graph meter, e.g. graph mode presented on display 1341, according to one embodiment. Recto-anal inhibitory reflex describes the relaxation of the internal anal sphincter in response to distention of the rectum. RAIR has been described above with respect to FIGS. 11, 17, 18, and 19. Graph 2405 shows a visual representation of what a graph meter would record if RAIR is present, i.e. a normal condition. Graph 2410 shows a visual representation of what a graph meter would record if RAIR is not present, i.e. an abnormal condition.

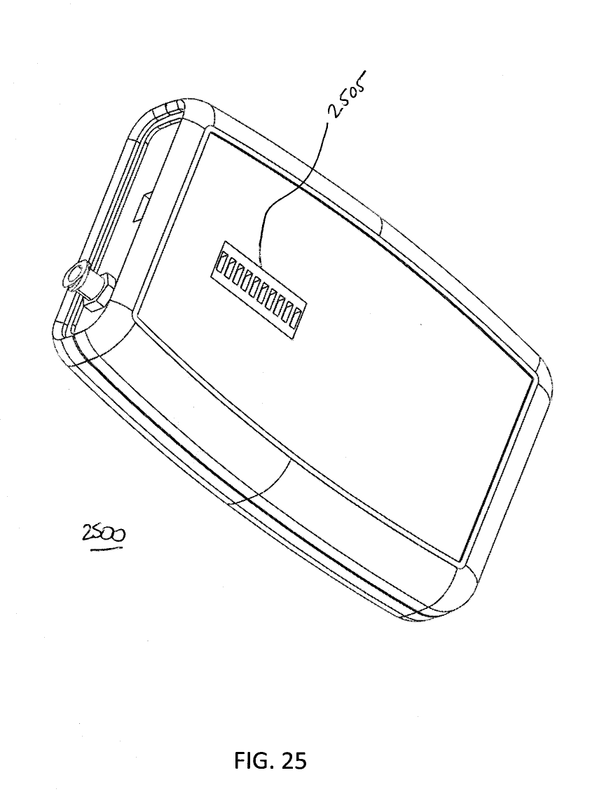

FIG. 25 is a top perspective view of a handheld device 2500 that provides biofeedback, according to one embodiment. The biofeedback is provided via an array of lights 2505 that illuminate in sequence as the Anorectal force increases. This device, similar to the devices depicted in FIGS. 2, 3, and 4, is intended for use by the patient at home after therapy with the physician. This device is used in conjunction with standard Anorectal probes commercially available.

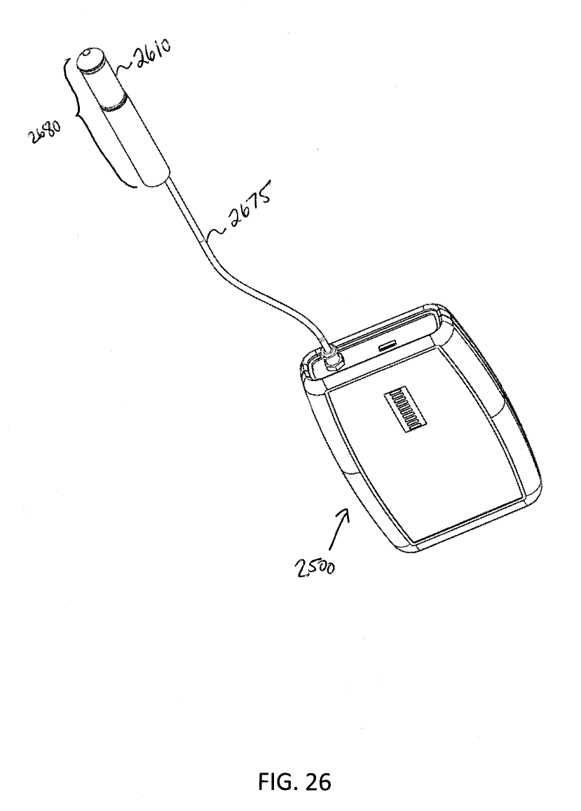

FIG. 26 is a perspective view of handheld device 2500 for use with a probe, according to one embodiment. Handheld device 2500 is connected to an electrical connection 2675. Electrical connection 2675 is in turn coupled to sensor 2610 of probe 2680. As stated above with respect to FIG. 25, probe 2680 may be a standard anorectal probe that is commercially available.

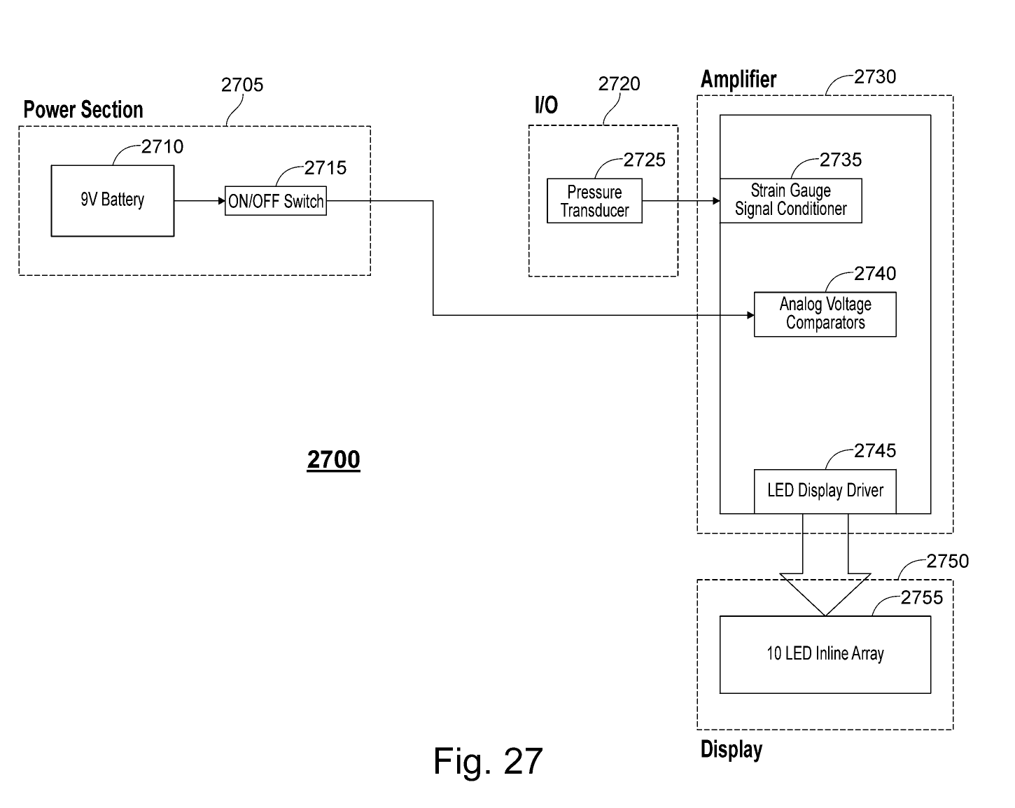

FIG. 27 is an electronics architecture 2700 according to one embodiment. Electronics architecture 2700 may comprise the internal electronics of handheld device 2500. Electronics architecture 2700 comprises a power section 2705, and input/output section 2720, an amplifier section 2730, and a display section 2750. Power section has a battery 2710 and an ON/OFF switch 2715. Battery 2710 may be a 9V battery or any other batter capable of providing power to device 2500, 2600, 2700. Power is provided to amplifier section 2730 via ON/OFF switch 2715. Amplifier section 2730 receives a pressure indication from pressure transducer 2725 via input/output section 2720. The pressure indication is received at amplifier 2730 via the strain gauge signal conditioner 2735. Amplifier 2740 also comprises analog voltage comparators 2740 and light emitting diode (LED) display driver 2745. LED display driver 2745 to display section 2750 in order to provide biofeedback to a user of the device depicted in FIG. 26. In one embodiment, display 2750 is a 10 LED inline array 2755.

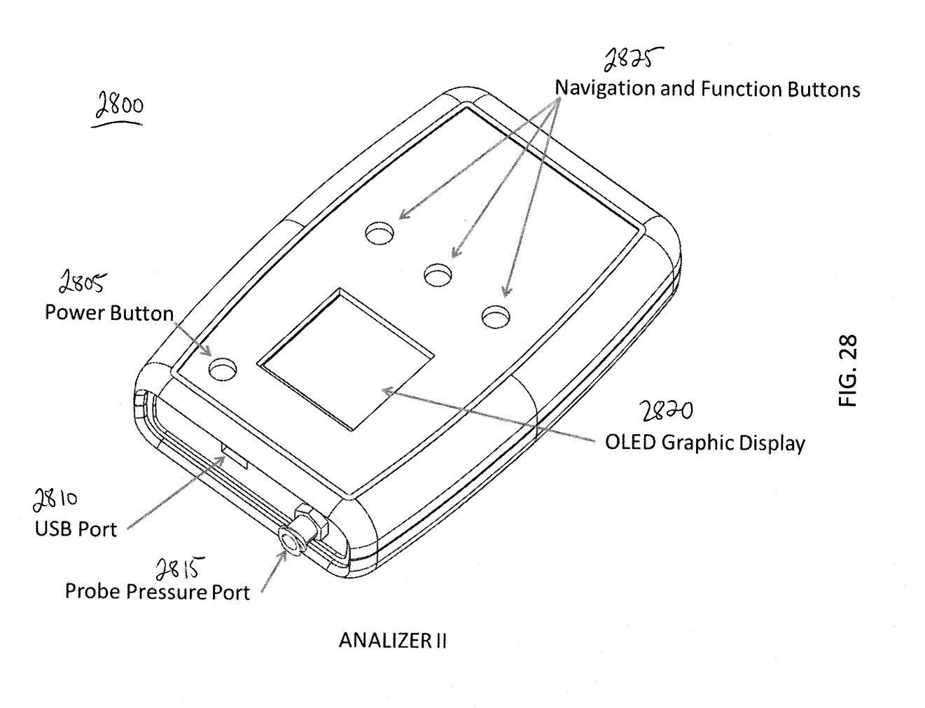

FIG. 28 illustrates a device 2800 for providing pelvic region diagnostic information, according to one embodiment. Device 2800 may be used in conjunction with analyzer 1300 as for example, pressure reading device 1340 and/or pressure reading device 1370. Device 2800 has a power button 2805, a USB port 2810, a probe pressure port 2815, a graphic display 2820, and navigation and function buttons 2825. In one embodiment, graphic display 2820 is an organic light emitting diode (OLED) display.

The present invention provides a simplification of the learning process required for the patient participation aspect of pelvic region rehabilitation and complements an electrical stimulation part of pelvic rehabilitation. Biofeedback is provided to the patient in response to their anorectal muscle action. A standard pelvic region probe, e.g. an anal or vaginal probe, is used to indicate, for example, anal sphincter muscle response. A bright colored display may provide easy to read and interpreted analog indication of muscle response. The device may be composed of battery powered analog electrical circuitry for simplicity and high reliability. A standard medical male luer connection for common probes may also be used.

The present invention provides a handheld anorectal manometry device. Anorectal manometry measures the pressure of anal sphincters in order to diagnose constipation and/or anal incontinence due to certain disorders. The present invention also provides a handheld rectoanal inhibitory reflex (RAIR) and handheld rectal sensation threshold, tone diagnostic test. The rectal sensation threshold, tone diagnostic test allows measures patient response to graded balloon distention. In addition, the present invention provides a handheld compliance test.

The present invention provides direct measurement of anal sphincter contraction pressure and provides direct measure of rectal balloon pressure. The present invention can determine and display, in digital and graphical form, a rectal compliance ratio. A direct measurement of anal probe pressure for RAIR, and resting and squeeze diagnosis may be obtained using the present invention.

The present invention provides a unique probe that in one embodiment, combines an anal balloon with an anorectal probe. This unique probe is alignment insensitive. The unique probe of the present invention allows the anal balloon to be extended away from the anorectal probe for compliance diagnosis. In addition, the probe of the present invention allows the anal balloon to be seated next to the anorectal probe for RAIR diagnosis.

Diagnostic data can be stored digitally for retrieval to a device display. Diagnostic data can be downloaded to an external computer, laptop, tablet, smart phone, or other computing device via USB or wireless technologies. Data analysis and report generation can be performed on external computers, laptops, tablets, smartphones, or other computing devices. A rechargeable battery is recharged through a USB connector to a computer or wall power adapter. A multifunction display presents diagnostic data in graphical and numeric formats.

The foregoing description and accompanying drawings illustrate the principles, exemplary embodiments, and modes of operation of the disclosure. However, the disclosure should not be construed as being limited to the particular embodiments discussed above. Additional variations of the embodiments discussed above will be appreciated by those skilled in the art and the above-described embodiments should be regarded as illustrative rather than restrictive. Accordingly, it should be appreciated that variations to those embodiments can be made by those skilled in the art without departing from the scope of the disclosure as defined by the following claims.

What is claimed is:

1. A pelvic region analyzer for performing at least one of therapeutic and diagnostic methods, comprising:
a probe for insertion in a pelvic region;
a balloon for insertion in the pelvic region and disposed at a distal end of the probe;
a sensor integrated with the probe and disposed at a distal end of the probe;
an electronic coupling coupled to the sensor, wherein the electronic coupling provides pressure information from the sensor; and
an air tube connected to the balloon, wherein air is added and removed from the air tube in accordance with at least one of the therapeutic and diagnostic methods.

2. A pelvic region analyzer, comprising:
a probe shaped to be inserted into a pelvic region;
a sensor integrated with the probe; and
an electronic coupling coupled to the sensor, the electronic coupling providing pressure information from the sensor in a form to be analyzed by a user.

3. The pelvic region analyzer according to claim 2, wherein the sensor is disposed at a distal portion of the probe.

4. The pelvic region analyzer according to claim 2, wherein the pressure information comprises at least one of a resting pressure and a squeeze pressure.

5. The pelvic region analyzer according to claim 2, wherein the pressure information comprises at least one of a visual textual display, a visual graphical display, and an aural display.

6. The pelvic region analyzer according to claim 2, further comprising:
a balloon shaped to be inserted into the pelvic region, the balloon disposed at a distal end of the probe; and
an air tube assembly fluidically connected to the balloon and operable to inflate and deflate the balloon selectively.

7. The pelvic region analyzer according to claim 5, wherein at least a portion of the air tube assembly is disposed at least one of:
within the probe; and
alongside the probe.

8. The pelvic region analyzer according to claim 6, wherein the air tube assembly comprises:
an air tube; and
a valve coupled to the balloon through the air tube and operable to prevent air from exiting out from the balloon.

9. The pelvic region analyzer according to claim 8, further comprising a pressure reading device coupled to the valve and operable to measure pressure exerted on the balloon.

10. The pelvic region analyzer according to claim 8, further comprising an inflation device fluidically coupled to the valve and operable to introduce air into the balloon through the valve.

11. The pelvic region analyzer according to claim 10, wherein the air tube is a first air tube, and further comprising a second air tube different from the first air tube and fluidically coupling the valve to the inflation device.

12. The pelvic region analyzer according to claim 11, further comprising a bifurcated joint fluidically coupling the first air tube to the second air tube.

13. The pelvic region analyzer according to claim 12, wherein the valve is:
fluidically coupled to the balloon through the first air tube, the second air tube, and the bifurcated joint; and
operable to prevent air from exiting out from the balloon.

14. The pelvic region analyzer according to claim 13, wherein the valve is a luer-lock two way valve.

15. The pelvic region analyzer according to claim 14, further comprising a pressure reading device coupled to the valve and operable to measure pressure exerted on the balloon.

16. The pelvic region analyzer according to claim 14, further comprising an inflation device fluidically coupled to the valve and operable to introduce air into the balloon through the valve.

17. A method for analyzing pelvic dysfunction, which comprises:
providing personal pelvic floor dysfunction therapy by:
receiving a pressure indication in response to a muscle squeeze of a user;
providing an indication of muscle squeeze strength;
providing an indication of muscle endurance; and
providing an indication of a successful muscle squeeze.

18. The method according to claim 17, which further comprises carrying out the muscle-squeeze-strength-indication step by indicating the muscle squeeze strength with at least one of a volume and a frequency of a sound.

19. The method according to claim 17, which further comprises carrying out the muscle-endurance-indication step by indicating the muscle endurance by a length of time that the sound lasts.

20. The method according to claim 17, which further comprises carrying out the successful-muscle-squeeze-indication step by indicating the successful muscle squeeze with at least one of an aural indication, a visual indication, and a vibratory indication.

21. A method for analyzing pelvic dysfunction, which comprises:
providing an anal manometry diagnostic test with a handheld diagnostic device by:
entering a pressure reading mode of the handheld diagnostic device;
placing a sensor of the handheld diagnostic device adjacent an internal anal sphincter muscle of a patient;
recording a highest resting pressure exerted by the internal anal sphincter muscle on the sensor; and
recording, with the handheld diagnostic device, a highest squeeze pressure exerted by the external anal sphincter muscle on the sensor.

22. A method for analyzing pelvic dysfunction, comprising:
providing a Recto-Anal Inhibitory Reflex (RAIR) diagnostic test using a handheld diagnostic device by:
entering a graphic mode of the handheld diagnostic device;
placing a sensor of the handheld diagnostic device adjacent an internal anal sphincter muscle of a patient;
determining a highest resting pressure exerted by the internal anal sphincter muscle on the sensor; and
measuring patient response to rapid introduction of air into, and rapid withdrawal of air out of, a balloon of the handheld diagnostic device.

23. A method for analyzing pelvic dysfunction, comprising:
providing a rectal sensation threshold tone and compliance diagnostic test using a handheld diagnostic device by:
entering a pressure reading mode of the handheld diagnostic device;
placing a balloon of the handheld diagnostic device into a rectal ampulla of a patient;
increasing air in the balloon until the patient responds to a first sensory threshold;
recording a pressure reading and a volume of air corresponding to the first sensory threshold;
increasing air in the balloon until the patient responds to a second sensory threshold;
recording a pressure reading and volume of air corresponding to the second sensory threshold; and
determining a rectal sensation threshold tone and compliance value based on at least one of the pressure reading corresponding to the first sensory threshold and the pressure reading corresponding to the second sensory threshold.
